(19) United States
(12) Patent Application Publication
(10) Pub. No.: US 2012/0184952 A1
(43) Pub. Date: Jul. 19, 2012

(54) LOW-PROFILE OFF-WALL ELECTRODE DEVICE FOR RENAL NERVE ABLATION

(76) Inventors: Mark L. Jenson, Greenfield, MN (US); Scott Smith, Chaska, MN (US)

(21) Appl. No.: 13/243,729

(22) Filed: Sep. 23, 2011

Related U.S. Application Data
(60) Provisional application No. 61/434,136, filed on Jan. 19, 2011.
(60) Provisional application No. 61/503,382, filed on Jun. 30, 2011.

(51) Int. Cl. A61B 18/14

(52) U.S. Cl. 606/41

(57) ABSTRACT

A catheter includes at least one electrode provided at its distal end. A spacing structure, provided at the catheter's distal end and encompassing the electrode, is transformable between a low-profile introduction configuration and a larger-profile deployed configuration, and maintains space between the electrode and a wall of a renal artery when electrical energy sufficient to ablate perivascular renal nerve tissue adjacent the renal artery is delivered by the electrode. The spacing structure may comprise perforations allowing for passage of arterial blood therethrough and transport of high frequency alternating current from the electrode to the perivascular renal nerve tissue via the blood, with no or negligible thermal injury to the artery wall. An ablation catheter with an electrode encompassed spacing structure can be deployed in each renal artery to deliver bipolar RF energy for ablating perivascular renal nerve tissue and ganglia near the aortorenal junctions.

### RELATED PATENT DOCUMENTS

This application claims the benefit of Provisional Patent Application Ser. Nos. 61/434,136, filed Jan. 19, 2010, and 61/503,382 filed Jun. 30, 2011, to which priority is claimed pursuant to 35 U.S.C. §119(e) and which are hereby incorporated herein by reference.

### SUMMARY

Devices, systems, and methods of the disclosure are directed to ablating target tissue of the body using an electrode arrangement that positions one or more electrodes a distance away from body tissue during ablation of the target tissue. Devices, systems, and methods of the disclosure are directed to ablating target tissue adjacent a body vessel, chamber, cavity, or tissue structure using an electrode arrangement that positions one or more electrodes a distance away from the body vessel, chamber, cavity, or tissue structure during ablation of the target tissue. Devices, systems, and methods are directed to denervating tissues that contribute to renal sympathetic nerve activity, such as perivascular renal nerves and ganglia at or near the aortorenal junction, using high frequency alternating current delivered from one or more electrode positioned a distance away from the inner wall of a renal artery during ablation. Ablation apparatuses and methods are directed to unipolar and bipolar electrode configurations.

Various embodiments of the disclosure are directed to ablation apparatuses and methods of ablation that include or use a spacing arrangement to maintain space between one or more electrodes of a unipolar or bipolar electrode arrangement and tissue of the body. The spacing arrangement is preferably configured to maintain positioning of one or more electrodes a short distance away from body tissue during an ablation procedure. Spacing arrangements can be implemented for centering an electrode within a body vessel or to maintain off-center positioning of an electrode during ablation. Although described in the context of ablation procedures performed from within a vessel hereinbelow, it is understood that positioning apparatuses consistent with the present disclosure may be implemented to maintain space between electrodes configured for RF unipolar or bipolar ablation and a body vessel, chamber, cavity, or tissue structure (e.g., organ) during ablation.

According to some embodiments, an ablation apparatus includes a catheter, a conductor arrangement provided along the catheter, and at least one electrode provided at a distal end of the catheter and in communication with the conductor arrangement. The apparatus further includes a spacing structure provided at the distal end of the catheter and encompassing the at least one electrode. The spacing structure is configured to transform between a low-profile introduction configuration and a larger-profile deployed configuration. The spacing structure is further configured to maintain space between the at least one electrode and a body vessel, chamber, cavity, or tissue structure when electrical energy sufficient to ablate target tissue adjacent the body vessel, chamber, cavity, or tissue structure is delivered by the at least one electrode. The spacing structure may comprise perforations allowing for passage of a body fluid therethrough and transport of high frequency AC energy from the at least one electrode to the body vessel, chamber, cavity, or tissue structure via the body fluid.

The catheter may be configured as an infusion catheter through which a fluid can be transported. Suitable fluids include fluids that facilitate one or more of reducing electrical conductivity of surrounding body fluid, reducing fouling of a surface of the at least one electrode by surrounding body fluid, cooling tissue adjacent the at least one electrode, or comprises imaging contrast media. In some embodiments, the spacing structure can be configured to center the at least one electrode within a body vessel when in the deployed configuration. In other embodiments, the spacing structure is configured to position the at least one electrode at an off-center location within a body vessel when in the deployed configuration.

In accordance with further embodiments, an ablation apparatus includes a first ablation apparatus configured for placement within a first renal artery and a second ablation apparatus configured for placement within a second renal artery. Each of the first and second ablation apparatuses comprise a catheter, a conductor arrangement provided along the catheter, at least one electrode provided at a distal end of the catheter and in communication with the conductor arrangement, and a spacing structure provided at the distal end of the catheter and encompassing the at least one electrode. The spacing structure is configured to transform between a low-profile introduction configuration and a larger-profile deployed configuration, and further configured to maintain space between the at least one electrode and a wall of the respective first and second renal arteries when in the deployed configuration. Each of the at least one electrode of the first and second ablation apparatuses cooperate as a bipolar electrode arrangement for delivering high frequency alternating current sufficient to ablate perivascular renal nerve tissue adjacent the first and second renal arteries and ganglia located at or near first and second aortorenal junctions. The apparatus may includes a sheath having a lumen dimensioned to receive the first and second ablation apparatuses and a length sufficient to deliver the first and second ablation apparatuses to a location at or proximate the first and second renal arteries.

Various embodiments are directed to ablation methods. In some embodiments, and for each of a patient's renal arteries, methods involve causing a support structure of an ablation apparatus situated within the artery to transform between a low-profile introduction configuration and a larger-profile deployed configuration, and positioning an electrode of the ablation apparatus within the artery using the support structure in the deployed configuration. Methods further involve ablating perivascular renal nerve tissue adjacent the renal arteries and ganglia located at or near the patient's aortorenal junctions using the electrodes in a bipolar configuration while the support structures are in the deployed configuration, and causing the support structures to transform from the larger-profile deployed configuration to the low-profile introduction configuration after ablation.

In some method embodiments, positioning the electrode involves positioning the electrode at a center location within the artery when the support structure is in the deployed configuration. In other embodiments, positioning the electrode involves positioning the electrode at an off-center location within the artery when the support structure is in the deployed configuration. Methods may also involve transporting a fluid through the ablation apparatus, the fluid facilitating one or more of reducing electrical conductivity of blood flowing near the electrodes, reducing fouling of a surface of the electrodes, cooling wall tissue of the renal arteries, or comprising imaging contrast media.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates computer modeling of heat distribution through a vessel wall generated by an RF electrode placed in contact with the inner wall of the vessel;

FIG. 5 illustrates computer modeling of heat distribution through the same vessel wall generated by an RF electrode placed in a non-contacting relationship with the inner wall of the vessel in accordance with various embodiments;

FIG. 6 shows a bipolar off-wall RF electrode arrangement deployed in a patient's renal artery and in the patient's aorta in accordance with various embodiments;

FIG. 7 shows a bipolar off-wall RF electrode arrangement deployed in each of a patient's renal arteries in accordance with various embodiments;

FIGS. 8 and 9 show a bipolar off-wall RF electrode arrangement deployed in a patient's renal artery and in the patient's aorta in accordance with various embodiments;

FIG. 10 shows an off-wall RF electrode arrangement of an ablation catheter in a relatively collapsed configuration within an external sheath or guide catheter in accordance with various embodiments;

FIG. 11 illustrates a first off-wall electrode arrangement of an ablation catheter expanded and deployed in a renal artery, and a second off-wall electrode arrangement in a relatively collapsed configuration within an external sheath or guide catheter in accordance with various embodiments;

FIG. 12 shows a bipolar off-wall RF electrode arrangement deployed in each of a patient's renal arteries in accordance with various embodiments;

FIG. 13A shows an off-wall RF electrode arrangement of an ablation catheter in a collapsed configuration in accordance with various embodiments;

FIG. 13B shows the off-wall RF electrode arrangement of FIG. 13A in an expanded configuration in accordance with various embodiments;

FIG. 14 shows a bipolar off-wall RF electrode arrangement deployed in each of a patient's renal arteries in a collapsed configuration in accordance with various embodiments;

FIG. 15 shows a unipolar off-wall RF electrode arrangement positioned in a patient's renal artery and in an expanded configuration in accordance with various embodiments;

FIG. 16A shows a unipolar off-wall RF electrode arrangement of an ablation catheter in a collapsed configuration in accordance with various embodiments;

FIG. 16B shows the unipolar off-wall RF electrode arrangement of FIG. 16A in an expanded configuration in accordance with various embodiments;

FIG. 17 shows a unipolar off-wall RF electrode arrangement positioned in a patient's renal artery and in a collapsed configuration in accordance with various embodiments;

FIG. 18 shows the unipolar off-wall RF electrode arrangement of FIG. 17 in an expanded configuration in accordance with various embodiments;

FIG. 19 shows a representative RF renal therapy apparatus in accordance with various embodiments;

FIG. 20 shows an off-wall RF electrode arrangement biased against a side of the inner wall of the renal artery in accordance with various embodiments; and

FIG. 21 shows an embodiment of an off-wall spacing arrangement and an ultrasound ablation device encompassed by the off-wall spacing arrangement in accordance with various embodiments.

### DETAILED DESCRIPTION

Embodiments of the disclosure are directed to apparatuses and methods for ablating target tissue of the body. Embodiments of the disclosure are directed to apparatuses and methods for ablating perivascular renal nerves for the treatment of hypertension. Embodiments of the disclosure are directed to bipolar RF electrode arrangements configured to maintain positioning of electrodes in a space-apart relationship relative to an inner wall of a vessel during renal nerve ablation.

Ablation of perivascular renal nerves has been used as a treatment for hypertension. The autonomic nervous system includes afferent and efferent nerves connecting the kidneys to the central nervous system. At least some of these nerves travel in a perivascular location along the renal arteries. The exact locations of these nerves can be difficult to determine, but there is typically one or more ganglia just outside the aorta, near the junction with the renal artery, and nerves running along the renal arteries, with one or more additional ganglia. The ganglia are variable in number, size, and position, and can be located at the aortorenal junction, or around towards the anterior aspect of the aorta, or farther down along the renal artery, and can be on any side of the renal artery.

Conventional treatment approaches typically use monopolar radiofrequency (RF) electrodes placed in the renal artery to ablate the perivascular nerves, but with risk of artery wall injury. To control injury to the artery wall, one approach is to ablate at discrete locations along and around the artery, which simply limits the arterial injury to multiple smaller locations. With this approach, high current density typically occurs in the tissue closest to the electrode contact region, causing preferential heating and injury to the artery wall at each of the discrete locations. Multiple discrete ablations also extend the procedure time.

Due to the limitations of artery wall heating, previous approaches cannot treat certain patients, such as those with short or multiple renal arteries. Also, previous approaches require larger electrodes to reduce current density and improve heat transfer for artery wall cooling. In some situations, a lower-profile device may be desired, to reduce vascular complications or to facilitate radial artery access. A better approach to ablating renal sympathetic nerves for treatment of hypertension is needed, especially targeting the renal ganglia and further reducing arterial injury, preferably with lower profile devices.

Embodiments of the disclosure are directed to apparatuses and methods for RF ablation of renal autonomic ganglia and nerves for the treatment of hypertension with reduced renal artery injury. Various embodiments of the disclosure employ a bipolar off-wall RF electrode configuration to more effectively ablate nerves and ganglia near the renal ostium, without renal artery injury. Some embodiments employ a unipolar off-wall RF electrode configuration to more effectively ablate renal nerves and ganglia without renal artery injury.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

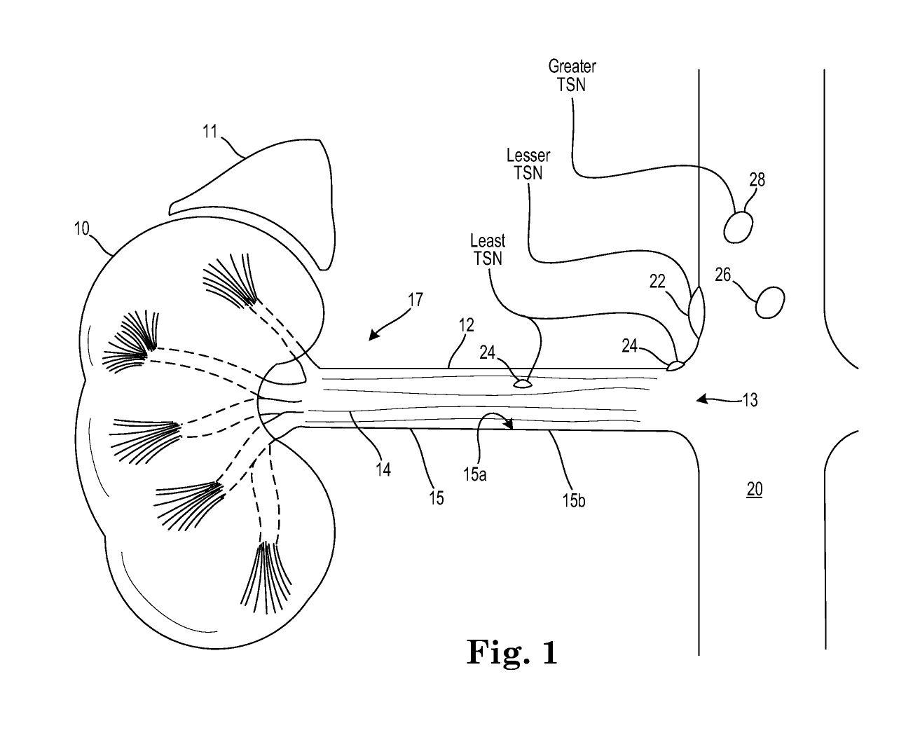

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

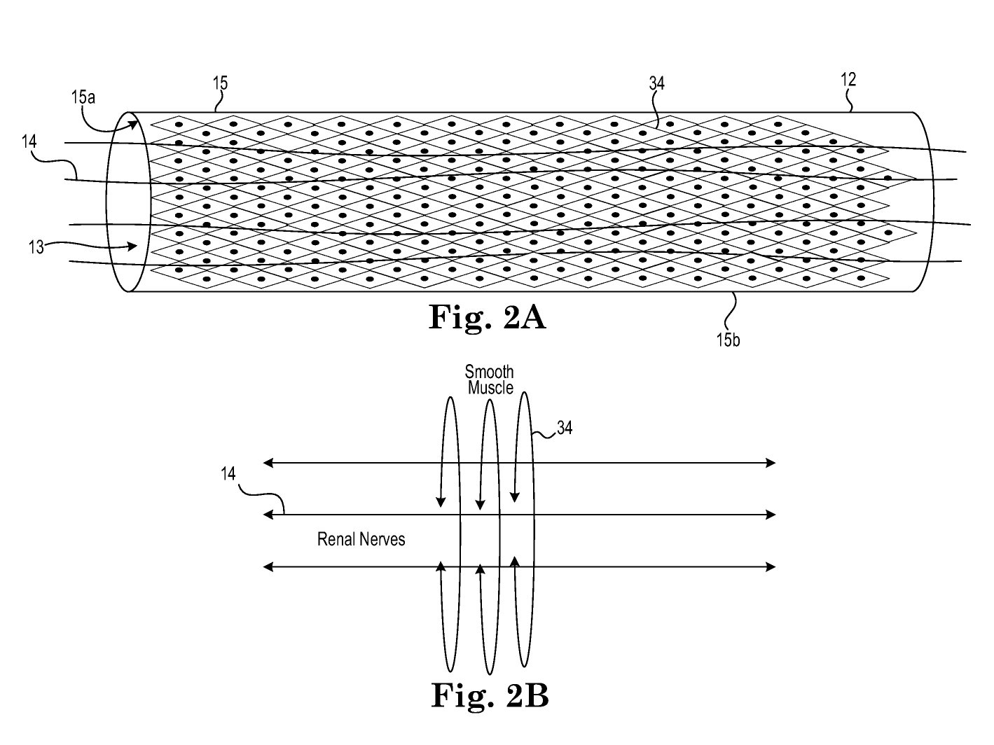

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

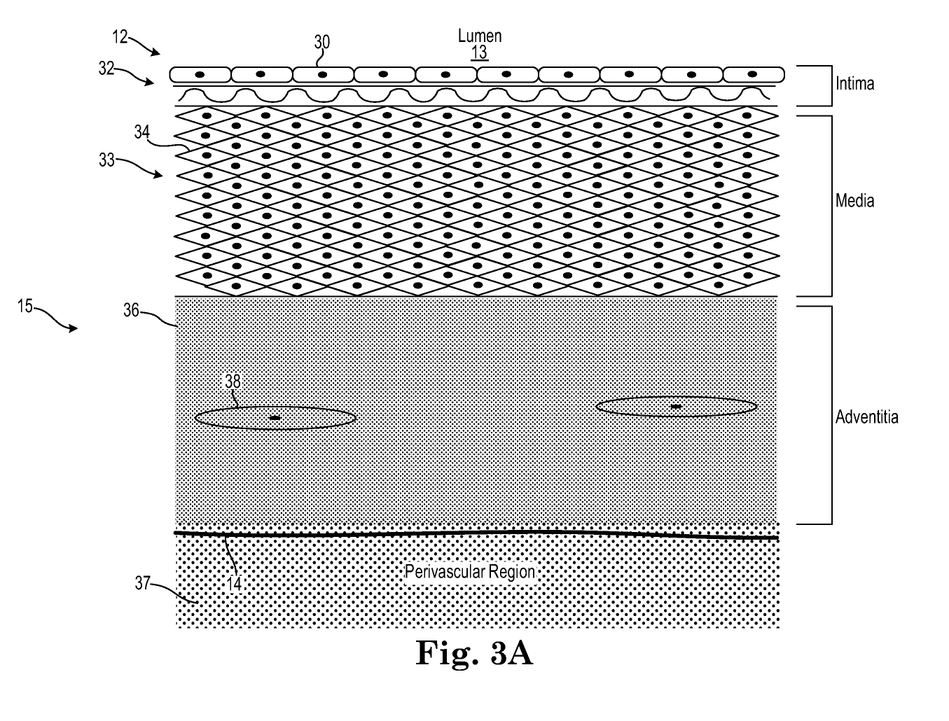

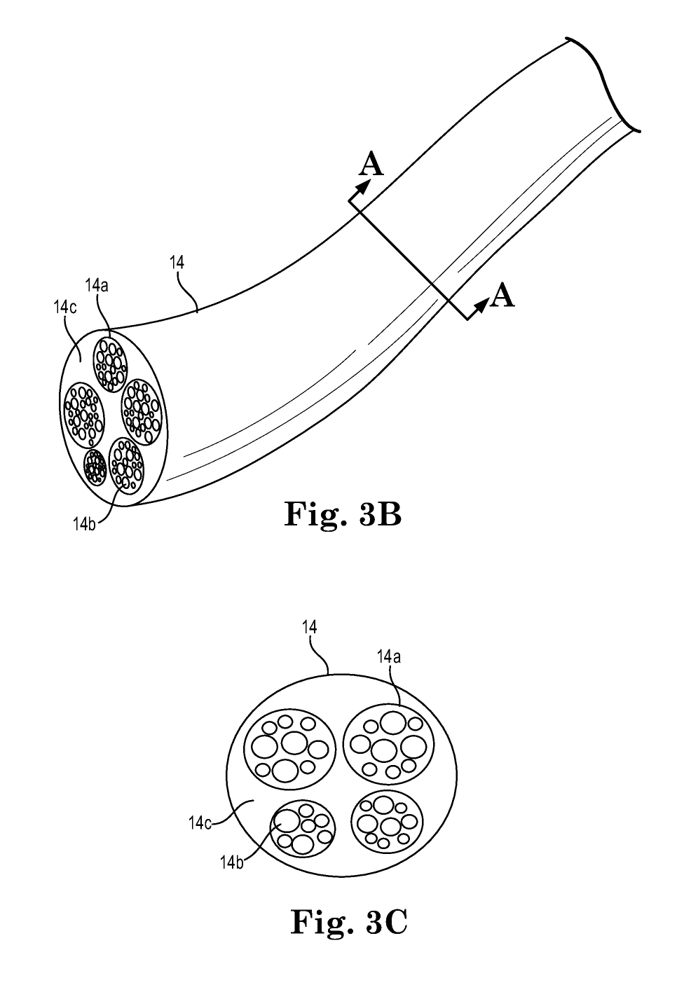

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b is preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

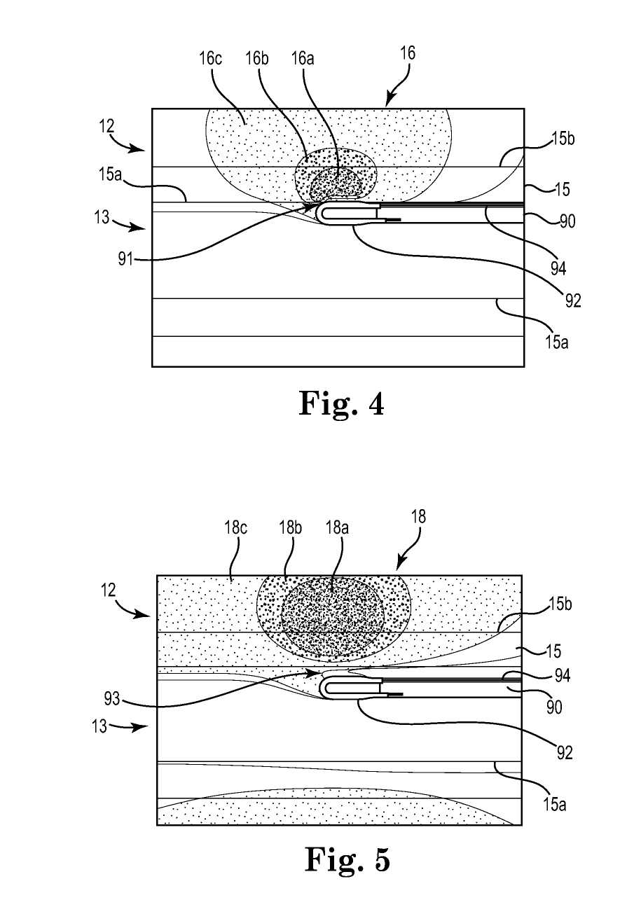

Turning now to FIGS. 4 and 5, there is illustrated computer modeling of heat distribution through a vessel wall generated by an RF electrode situated within the lumen of the vessel. In FIG. 4, the electrode is situated in direct contact with the inner wall of the vessel. In FIG. 5, the electrode is situated in a non-contacting (off-wall) relationship with respect to the inner wall of the vessel in accordance with various embodiments. It is readily apparent that temperatures of the vessel's inner wall resulting from the non-contact electrode-to-tissue interface shown in FIG. 5 are significantly lower than vessel inner wall temperatures resulting from the direct contact electrode-to-tissue interface of FIG. 4.

FIG. 4 shows the heat distribution profile 16 within the renal artery wall 15 produced by an ablation electrode 92 of a catheter 90 placed in direct contact with the vessel's inner wall 15a. The catheter 90 includes a conductor 94 that runs the length of the catheter and is connected to the electrode 92 situated at the distal end of the catheter 90. In the configuration shown in FIG. 4, the relative positioning of the electrode 92 and inner wall 15a of the renal artery wall 15 defines a direct contact electrode-to-tissue interface 91.

The heat distribution profile 16 of FIG. 4 demonstrates that the artery wall tissue at or nearest the artery's inner wall 15a is subject to relatively high temperatures. As can be seen in FIG. 4, heat produced by the electrode 92 at the direct contact electrode-to-tissue interface 91 is greatest at the inner wall 15a and decreases as a function of increasing distance away from the electrode 92. For example, a heating zone 16a associated with the highest temperatures produced by the ablation electrode 92 extends nearly the entire thickness of the artery wall 15. A zone 16b of lower temperatures relative to zone 16a extends radially outward from zone 16a, beyond the outer wall 15b of the renal artery 12, and into perivascular space adjacent the renal artery 12. A relatively cool zone 16c is shown extending radially outward relative to zones 16a and 16b. In this simulation, zone 16a is associated with temperatures required to ablate tissue which includes renal arterial wall tissue and perivascular renal nerves.

FIG. 5 shows the ablation electrode 92 of catheter 90 situated in a spaced-apart relationship relative to the inner wall 15a of the renal artery 12. In the configuration shown in FIG. 5, the relative positioning of the electrode 92 and inner wall 15a of the renal artery wall 15 defines a non-contact electrode-to-tissue interface 93. As can be seen in FIG. 5, the heat distribution profile 18 differs significantly from that shown in FIG. 4.

Importantly, zone 18a, which is associated with the highest temperatures (ablation temperatures), is translated or shifted outwardly away from the inner wall 15a and towards the outer wall 15b and perivascular space adjacent the renal artery 12. Zone 18a encompasses an outer portion of the adventitia of the renal artery wall 15 and encompasses a significant portion of the perivascular space adjacent the renal artery 12. As such, renal nerves and ganglia included within the adventitial tissue and perivascular space are subject to ablative temperatures, while the endothelium at the inner wall 15a of the renal artery 12 is maintained at a temperature which does not cause permanent injury to the inner wall tissue.

Off-wall electrode configurations according to various embodiments can reduce the RF current density in the artery wall 15, as the current spreads out somewhat as it passes between the electrode 102 and the artery wall 15 through the blood. This provides a sort of fluidic “virtual electrode” and results in less heating of the artery wall 15 due to the lower current density. According to various embodiments, structures that hold one or more electrodes at a prescribed distance away from the artery wall 15 preferably provide for passive cooling of the artery wall 15 during ablation by blood flowing through the artery 12. Separating the electrode(s) from the artery wall 15 by a structure that allows blood to pass along the artery wall 15 provides more effective cooling of the artery wall 15 (and the electrode 102), further reducing thermal injury to the artery wall 15. The current density in the target perivascular tissue can also be somewhat decreased, but the cooler artery wall temperatures allow greater overall power to be delivered safely, in order to achieve sufficient current density in the target tissue to ablate the target tissue.

Various embodiments of the disclosure are directed to apparatuses and methods for RF ablation of perivascular renal nerves for treatment of hypertension, employing one or more bipolar off-wall RF electrode configurations to more effectively ablate renal nerves and ganglia near the renal ostium, while avoiding injury to the renal artery. A bipolar off-wall RF electrode arrangement of the disclosure includes multiple electrodes held slightly away from the artery and/or aortal wall, resulting in decreased current density and improved cooling from the blood to reduce arterial and/or aortal injury while maintaining target tissue at ablation temperatures.

According to some embodiments, an off-wall electrode arrangement maintains positioning of one or more electrodes at a separation distance ranging from about 0.5 mm to about 3 mm away from a vessel wall. According to other embodiments, an off-wall electrode arrangement maintains positioning of one or more electrodes at a separation distance ranging from about 1 mm to about 1.5 mm away from a vessel wall. Prior approaches have used an RF electrode placed in direct contact with the renal artery, for example, but have had difficulty in repositioning the electrode to complete ablation while minimizing injury to the artery wall due to peak current density and heating at the wall contact points.

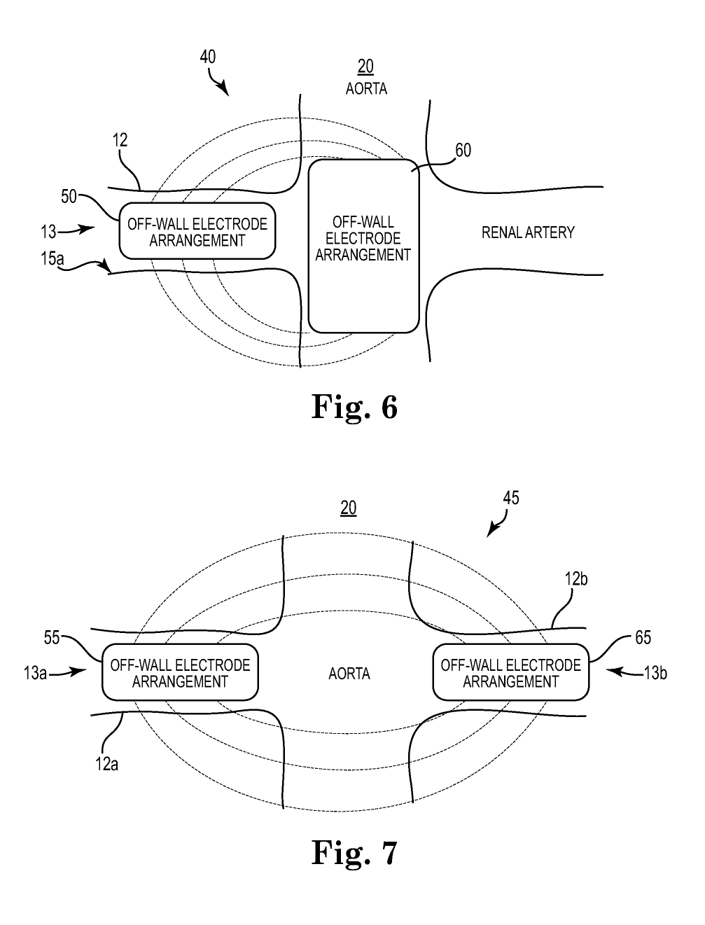

With reference to FIG. 6, a bipolar off-wall RF electrode arrangement is shown deployed in a patient's renal artery 12 and in the patient's aorta 20. The bipolar electrode arrangement 40 shown in FIG. 6 includes an electrode arrangement 50 deployed in the lumen 13 of the renal artery 12 and an electrode arrangement 60 deployed in the aorta 20 at a location near the aortorenal junction. Each of the electrode arrangements 50 and 60 includes one or more RF electrodes which are maintained a predefined distance away from the renal artery wall and the aortal wall, respectively. The electrode arrangements 50 and 60 are configured to operate as a bipolar RF electrode configuration.

FIG. 7 shows another embodiment of a bipolar off-wall RF electrode arrangement. In the embodiment illustrated in FIG. 7, the bipolar electrode arrangement 45 includes an electrode arrangement 55 deployed in the lumen 13a of one of the patient's renal arteries 12a. A second bipolar electrode arrangement 65 is shown deployed in a lumen 13b of the patient's other renal artery 12b. Each of the electrode arrangements 55 and 65 includes one or more RF electrodes which are maintained a predefined distance away from the respective renal artery walls. The electrode arrangements 50 and 60 are configured to operate as a bipolar RF electrode configuration.

It is noted that, in some embodiments, a ground pad may be used in the configurations shown in FIGS. 6 and 7. For example, bipolar ablation may be performed using selected electrodes of one or both of the bipolar electrode arrangements shown in the Figures and the ground pad. For configurations that include a ground pad, renal denervation may be selectively performed in a unipolar ablation mode or a bipolar ablation mode. It is further noted that the ablation zone is close to the electrode in a unipolar ablation configuration, but also close to the two electrode arrangements in a bipolar ablation configuration. Also, it is understood that the electric field strength decreases as a function of the square of distance from the electrodes.

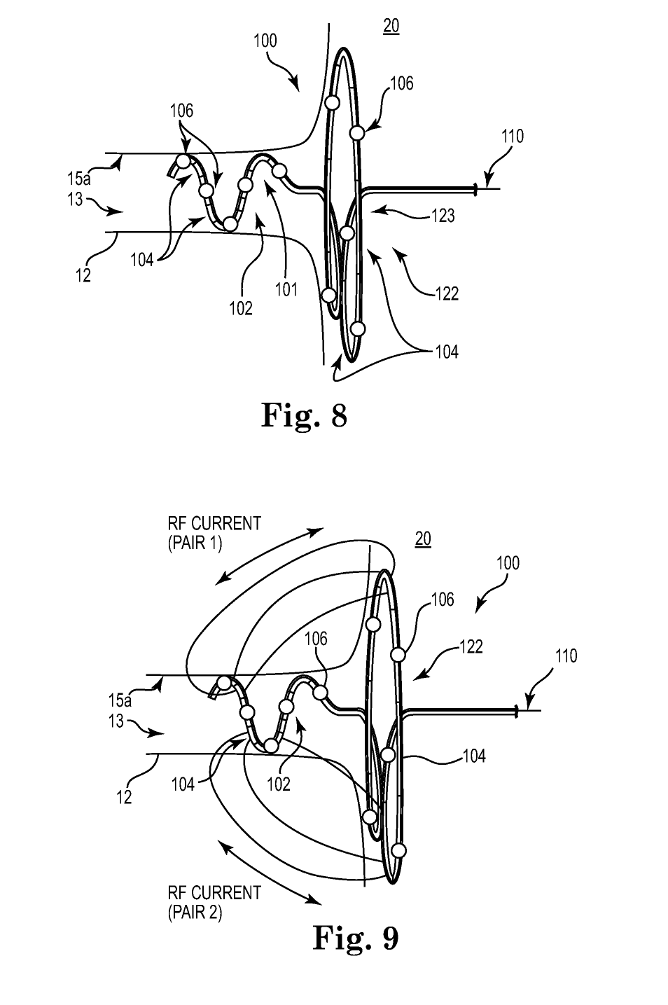

According to various embodiments, and as illustrated in FIGS. 8 and 9, an RF ablation catheter 100 includes a first electrode arrangement 102 with at least one and preferably multiple electrodes 104 mounted on a first expandable structure 101 to position the electrodes 104 near, but not in direct contact with, the inner artery wall 15a. Spacing features 106 hold the electrodes 104 a controlled distance from the renal artery wall 15a for effective wall cooling and to decrease current density at the artery wall 15a.

A second electrode arrangement 122 is incorporated into the same catheter, or into a modified guide catheter or sheath, similarly positions electrodes 104 near the wall of the aorta 20. The second electrode arrangement 122 includes at least one and preferably multiple electrodes 104 mounted on a second expandable structure 123 to position the electrodes 104 near, but not in direct contact with, the inner wall of the aorta 20 proximate the aortorenal junction. Spacing features 106 maintain the electrodes 104 at a controlled distance from the aortal wall for effective wall cooling and to decrease current density at the aortal wall.

Bipolar activation by an external control unit passes RF energy between aortic and renal artery electrodes 104 of the first and second electrode arrangements 102, 122 to preferentially ablate perivascular tissue near the renal artery ostium where significant autonomic ganglia are typically located. In some embodiments, an optional helical actuation wire 110 can be provided within a lumen of the ablation catheter 100. The helical actuation wire 110 can be displaced in a distal or proximal direction to selectively collapse and expand the first and second expandable structures 101 and 123 of the ablation catheter 100.

FIG. 9 schematically illustrates RF current passing between two different pairs of electrodes 104 mounted on the first and second expandable structures 101, 123, and passing through the target tissue. Control of which electrodes 104 are energized and in what combinations and timing is determined automatically by a control unit which supplies RF energy to the electrodes 104. For example, selected electrodes 104 of each of the first and second electrode arrangements 102 can be activated to define or contribute to different RF current paths. Bifurcated or multiple renal artery anatomies can be treated with this approach as well. By control of which electrodes 104 are active, switching between electrodes 104, and positioning the electrodes 104 away from the vessel walls, effective heating of the perivascular tissue containing significant nerves and ganglia is achieved while minimizing thermal injury to the renal artery 12 and the aorta 20.

Each electrode 104 in the first electrode arrangement 102 has a corresponding insulated conductor to connect to the external control unit. The control unit energizes electrodes 104 of the first and second electrode arrangements 102 and 122 in a prescribed pattern and sequence. Monitoring of the tissue impedance between various electrode pairs offers improved evaluation of the extent of tissue ablation. It is understood that some of the electrodes 104 in the first electrode arrangement 102 can be coupled in series if desired.

RF current passes between an electrode 104 in the renal artery 12 and an electrode 104 in the aorta 20, passing through the blood for a short distance before passing through the vessel walls and the intervening tissue. Since blood effectively cools the vessel wall, the target tissue is ablated without injury to the vessel walls. An infusion of fluid into the vessel(s) can reduce the conductivity of the blood to reduce current flow directly through the blood so that current preferentially passes through target tissues. A fluid infusion can also reduce effects on the blood and potential fouling of the electrode surface, allowing smaller electrodes to be used.

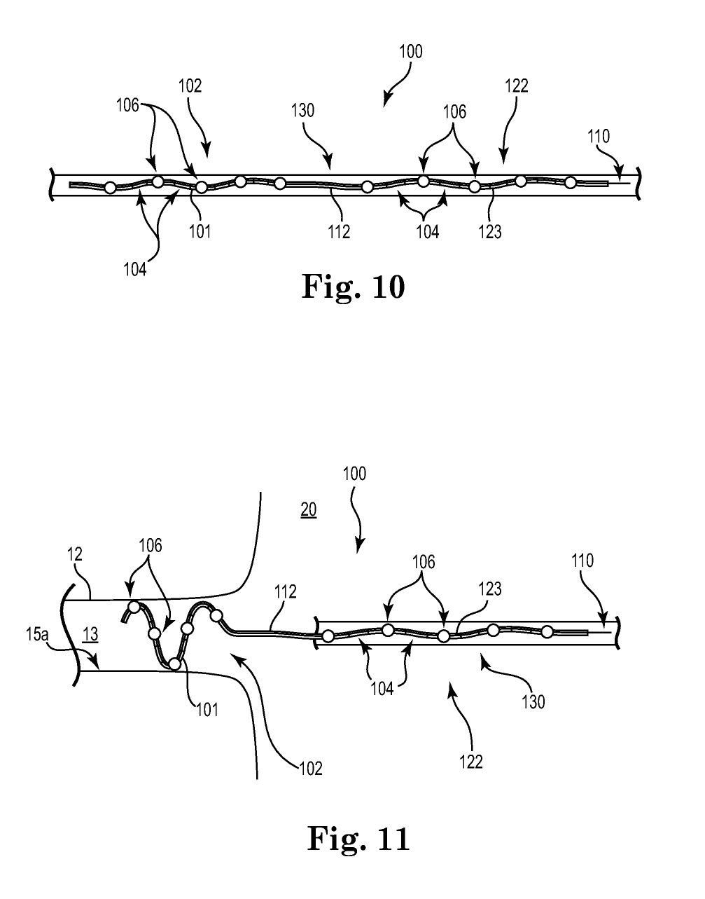

As is shown in FIGS. 10 and 11, the ablation catheter 100 has a low-profile introduction configuration. FIG. 10 shows the distal end of the ablation catheter 100 in a relatively collapsed configuration within an external sheath or guide catheter 130. The flexibility of the first and second electrode arrangement 102 and 122 of the ablation catheter 100 provides for enhanced navigation and advancement of the catheter 110 through the patient's vasculature.

In some embodiments, the first and second expandable structures 101 and 123 incorporate a shape-memory or a superelastic member configured to assume desired shapes when in their respective expanded configurations, such as those shown in FIGS. 8 and 9, for example. For example, the first and second expandable structures 101 and 123 can each incorporate a shape-memory or a superelastic helical wire. The helical wire of the first expandable structure 101 has a first diameter when assuming its deployed configuration. The helical wire of the second expandable structure 123 has a second diameter when assuming its deployed configuration. As can be seen in the embodiment illustrated in FIGS. 8 and 9, the second diameter is greater than the first diameter. In some embodiments, the second diameter is greater than the first diameter by a factor of at least 1.5. In other embodiments, the second diameter is greater than the first diameter by a factor of at least 2.

Although shown as a continuous unitary member in FIGS. 8 and 9, separate shape-memory or superelastic members may be used for each of the first and second expandable structures 101 and 123. A common sheath or separate sheaths may be used to deliver separate first and second expandable structures 101 and 123 to the renal artery 12 and aorta 20, respectively.

A transition region 112 may be defined between the separate shaping members, and include a material or component that facilitates independent movement of the separate members during expansion and collapsing. In some configurations, a continuous shape-memory or superelastic member can be fashioned with distal and proximal sections configured to assume desired shapes when in their respective expanded configurations.

In FIG. 11, the first electrode arrangement 102 is shown expanded and deployed in the renal artery 12. The first electrode arrangement 102 is preferably self-expanding, in that it transforms from its introduction configuration, shown in FIG. 10, to its deployed configuration, shown in FIG. 11, when the external sheath or guide catheter 100 is retracted from the first electrode arrangement 102. The spacing features 106 keep the electrodes 104 a short distance away from the artery wall. A variety of spacing features 106 can be utilized, including bumps or curves, struts or baskets, spherical or cylindrical elements, and the like. The spacing features 106 are chosen to minimize interference with blood flow past the artery wall 15a to maximize the cooling effect on the artery wall 15a.

FIG. 11 further shows the second electrode arrangement 122 about to expand as the sheath 130 is retracted. When in its expanded configuration, the spacing features 106 of the second electrode arrangement 122 keep the electrodes 104 a short distance away from the aorta wall 20. The perivascular tissues near the aortorenal ostium are ablated, including the target renal nerves and ganglia in that region. After completion of the ablation procedure, the first and second electrode arrangement 102 and 122 are collapsed, such as by advancing an external sheath 130 over the arrangements 102, 122. The ablation catheter 100 may be manipulated to advance the first electrode arrangement 102 into the contralateral renal artery 12, and the procedure may be repeated.

According to some embodiments, the first electrode arrangement 102 can incorporate a single electrode 104, with a positioning arrangement configured to hold the electrode 104 near the center of the renal artery 12. Rather than having the first and second electrode arrangements 102 and 122 on the same catheter, the second electrode arrangement 122 can be incorporated into the external sheath, guide catheter, or other device to provide more flexibility in positioning electrodes 104 of the second electrode arrangement 122. Multiple electrodes 104 of the first electrode arrangement 102 can be energized in parallel, and multiple electrodes 104 in the second electrode arrangement 122 can be energized in parallel, in a bipolar arrangement between first and second electrode arrangements 102 and 122. The second electrode arrangement 122 can be configured to deploy electrodes 104 at the opposite side of the aorta 20, or all around the aorta 20.

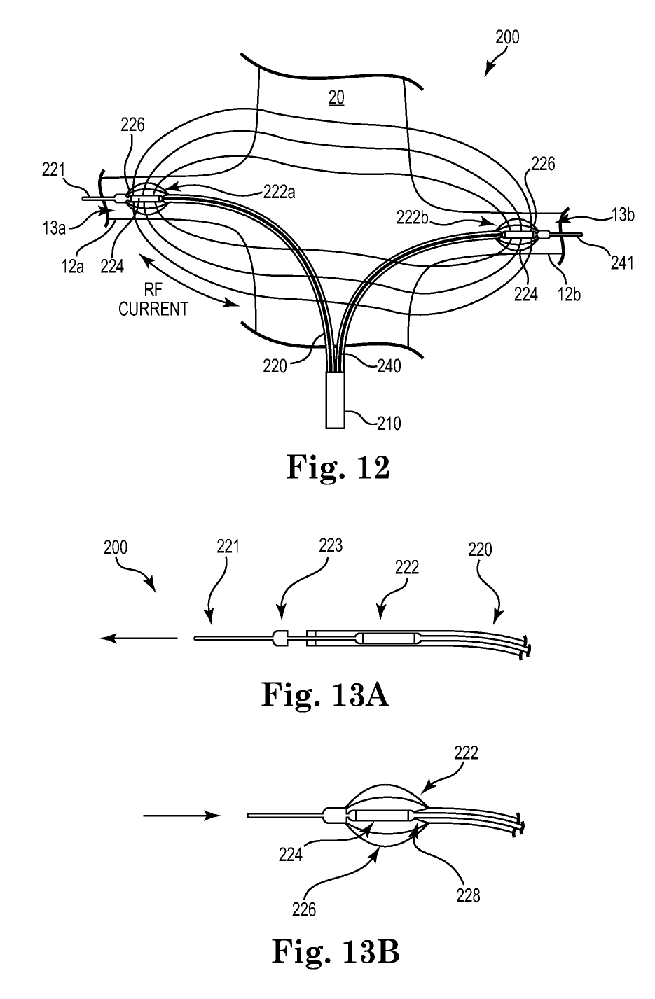

In accordance with various embodiments, apparatuses and methods are directed to bipolar RF ablation of renal autonomic ganglia and nerves with reduced renal artery injury using dual ablation catheters. Embodiments according to FIG. 12-14 use off-wall RF electrodes in each renal artery 12a and 12b to quickly and effectively ablate renal sympathetic nerves and ganglia without renal artery injury.

In the embodiments shown in FIGS. 12-14, a sheath 210 is shown positioned in the aorta 20 inferior to the aortorenal junction. It is understood that the sheath 210 may alternatively be positioned superior to the aortorenal junction. The sheath 210 has a lumen through which two ablation catheters 220 and 240 are advanced into respective renal arteries 12a and 12b. According to some embodiments, the sheath 210 has a diameter of about 6 French (Fr.) and each of the ablation catheters 220 and 240 has a diameter of about 3 Fr. In some embodiments, the ablation catheters 220 and 240 are configured as infusion catheters, allowing for imaging contrast injection into the renal arteries 12a and 12b.

Each of the ablation catheters 220 and 240 includes an RF electrode 224 encompassed by a centering basket 226. In a deployed configuration, as shown in FIG. 12, each centering basket 226 expands radially and makes contact with discrete circumferential locations of the respective inner renal artery walls. The centering baskets 226 are configured to position the RF electrode 224 preferably at a center location within the lumens 13a, 13b of the respective renal arteries 12a, 12b.

As is shown in FIGS. 12 and 14, a first electrode arrangement 222a is advanced into one renal artery 12a and a second electrode arrangement 222b is advanced into the other renal artery 12b. Each centering basket 226 is expanded to hold its respective electrode 224 a predetermined distance from the renal artery walls to ensure effective wall cooling from blood flow, and to decrease current density at the artery walls. It is noted that the first and second electrode arrangements 222a and 222b may be provided at the distal portions of a branched catheter, or two small separate catheters can be used.

Bipolar activation by an external control unit passes RF energy between the right and left renal artery electrodes 224 to preferentially ablate perivascular tissue near the renal artery ostium where significant autonomic ganglia are typically located. By positioning the electrodes 224 away from the vessel walls, the perivascular tissue is effectively heated while minimizing thermal injury to the renal artery and the aorta. FIG. 12 schematically illustrates RF current passing between electrodes 224 positioned in both renal arteries 12a and 12b, and passing through the target tissue. The control unit automatically controls the energizing of the electrodes 224. Since more effective heating of a larger amount of perivascular tissue is obtained without injury to the renal arteries 12a and 12b, even bifurcated or multiple renal artery anatomies may be treatable with this approach.

According to some embodiments, guidewires 221, 241 are provided to aid in positioning the first and second electrode arrangements 222a and 222b in the renal arteries 12a and 12b. The guidewires 221, 241 may have limited freedom to move with respect to the ablation catheters 220 and 140, so a curved wire tip can be employed and manipulated as needed to gain access to the renal arteries 12a and 12b. When configured as infusion catheters, ablation catheters 220 and 240 can be used for imaging contrast injection.

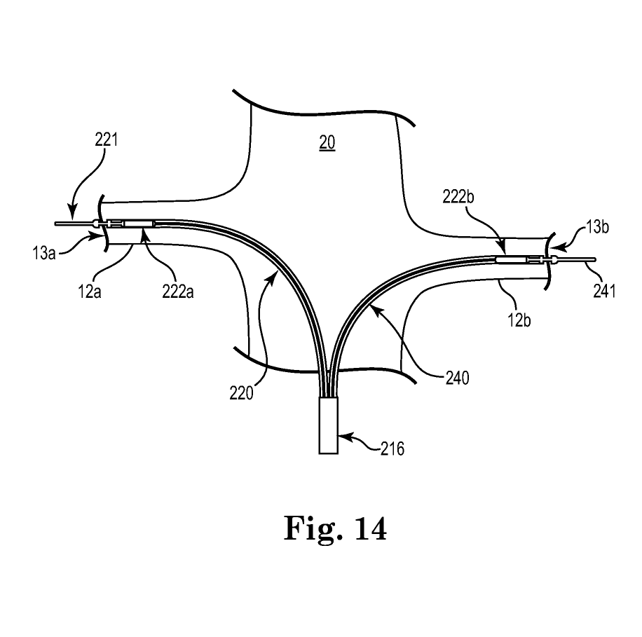

As can be seen in FIGS. 13a and 13b, the ablation catheters 220 and 240 have a low-profile introduction configuration. For simplicity of explanation, reference will be made to ablation catheter 220 in the following discussion, understanding that the description is equally applicable to ablation catheter 240. FIG. 13a shows a captured guidewire 221 and basket stop actuation of a collapsible centering basket 226. In the electrode arrangement 222, the electrode 224 is attached to the infusion catheter 220 by an insulated strut structure 228, which also provides for electrical power from the external control unit. The centering basket 226 is either non-conductive or is insulated, and preferably includes perforations or gaps which allow for passage of a conductive body fluid therethrough and transport of high frequency AC energy from the electrode 224 to adjacent tissue via the conductive body fluid. The centering basket 226 can be constructed as a self-collapsing structure. The centering basket 226 can incorporate a shape-memory or a superelastic member configured to assume a desired shape.

A basket actuation stop 223 is attached to the guidewire 221. After positioning the guidewire 221 as desired and advancing the ablation catheter 220 to the treatment position, the guidewire 221 and basket actuation stop 223 are retracted to actuate the centering basket 226 (by axial shortening and radial expansion) and maintain the basket 226 in a deployed configuration. The electrode ends can be insulated to avoid current concentrations near the ends of the electrode 224. After treatment, the guidewire 221 is advanced to allow the centering basket 226 to collapse (by axial lengthening and radial contraction). A sheath 210 (shown in FIG. 12) can be used to further collapse the centering basket 226 if needed. Alternatively, collapsing and expanding the centering basket 226 can be achieved by advancing and retracting a sheath over and from the centering basket 226. The centering basket 226 can be preferentially closed according to some embodiments (e.g., a self-collapsing structure).

In some embodiments, a somewhat larger basket configuration can be utilized that is self-expanding (but not necessarily self-collapsing), such as by use of an external sheath or a pull wire to collapse the centering basket 226. In other embodiments, the centering basket 226 need not be biased for self-expansion or self-collapsing, but may be push-pulled actuated or actuated using some combination of push, pull, and/or sheath arrangements.

FIG. 14 shows ablation catheters 222a and 222b in a collapsed configuration deployed respectively within the patient's left and right renal arteries 12a and 12b. Access to the lumen 13a and 13b of the left and right renal arteries 12a and 12b is facilitated by manipulation of guide wires 221 and 241. Having accessed the left and right renal arteries 12a and 12b using guidewires 221 and 241, ablation catheters 220 and 240 are advanced over their respective guide wires 221 and 241 and into renal arteries 12a and 12b using an over-the-wire technique. Proper positioning of the electrode arrangements 222a and 222b may be facilitated using imaging contrast injection into the ablation catheters 220a and 220b. In some embodiments, a radiopaque marker band can be provided at one or more locations of the ablation catheters 220 and 240 to enhance imaging of catheter positioning. With the electrode arrangements 222a and 222b positioned at desired locations within the renal arteries 12a and 12b, the captured guidewire 221 is pulled in a proximal direction toward the basket actuation stop 223. As discussed above, retraction of the guidewire 221 forces the centering basket 226 to compress longitudinally and expand radially into its deployed configuration, as is shown in FIG. 12.

An external control unit energizes the electrodes 204 of the electrode arrangements 222a and 222b in a bipolar manner. Monitoring of the tissue impedance between the electrodes 204 can be used for evaluation of the extent of tissue ablation. RF current passes between the electrodes 204 in the renal arteries 13a and 13b, passing through the blood for a short distance before passing through the vessel walls and the intervening tissue. Since blood effectively cools the vessel wall, the target tissue is ablated without injury to the vessel walls. An infusion of fluid through the ablation catheters 220 and 240 can locally reduce the conductivity of the blood to reduce current flow directly through the blood so that current preferentially passes through target tissues. As previously discussed, fluid infusion can also reduce effects on the blood and potential fouling of the electrode surface, allowing smaller electrodes to be used. The fluid may comprise imaging contrast media and/or contain an agent (e.g., cool saline) for cooling the vessel wall during ablation.

According to some embodiments, the infusion ablation catheters 220 and 240 can be D-shaped to maximize infusion space. Other electrode configurations can be used, including multiple electrodes in each renal artery. Spacer configurations other than the illustrated centering basket 226 can be used to keep the electrodes a minimum distance from the artery walls. In some embodiments, for example, a spacing structure can be constructed as a flexible structure, such as a slotted tube or inflatable balloon (preferably a porous balloon or a weeping catheter), capable of transforming between a low-profile (e.g., non-deployed) introduction configuration and a larger profile deployed configuration. When assuming a low-profile non-deployed configuration, the spacing structure facilitates delivery of the ablation arrangement to target tissue of the body, such as by way of femoral vasculature or through body tissue (e.g., thoracic tissue) from a percutaneous access location. Embodiments of a low-profile ablation device can also be used with radial artery access.

One electrode arrangement can be incorporated into a small infusion catheter similar to those shown, with the other electrode arrangement incorporated into an external sheath, guide catheter, or other device, to provide more flexibility in positioning the ablation regions or to improve profile or contrast injection capacity. A separate ground can be provided, such as with conventional skin ground pads or conductive portions of a guide catheter or sheath. Instead of or in addition to the bipolar RF configuration as shown, unipolar configurations with the ablation electrode(s) and the separate ground can be utilized.

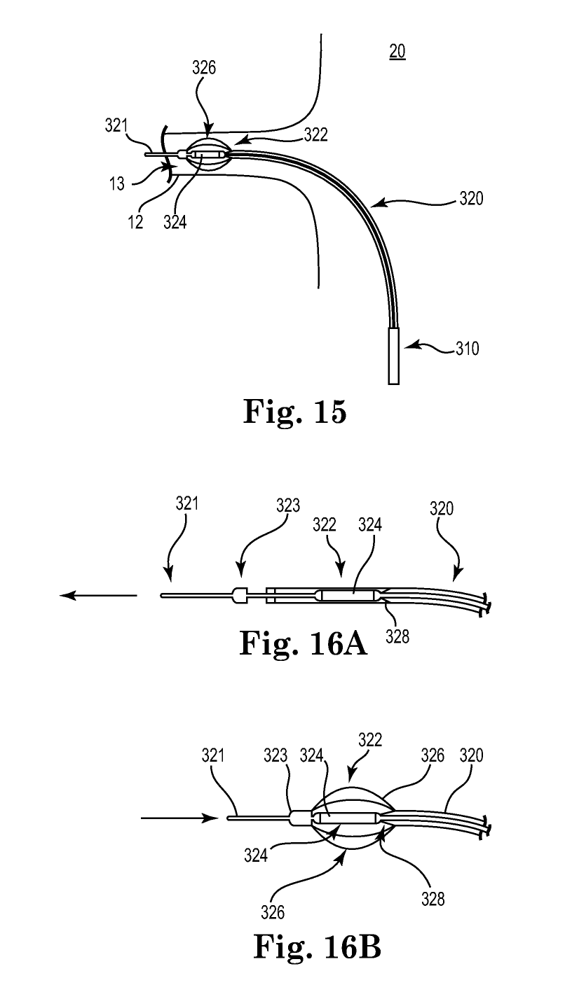

Turning now to FIGS. 15-18, there is shown an embodiment of an ablation catheter 320 configured for ablating renal nerves using a unipolar configuration. Embodiments according to FIGS. 15-18 use a low-profile device with an off-wall RF electrode in the renal artery to quickly and effectively ablate renal sympathetic nerves and ganglia without renal artery injury. This approach allows use of a relatively small (as compared to conventional access approaches) access sheath to reduce femoral vascular complications, and can also be used with radial artery access.

The ablation catheter 320 includes features similar to those previously described. Because the ablation catheter 320 is configured for individual deployment as compared to the dual ablation catheter configurations shown in FIGS. 12-14, somewhat larger components may be used if desired. For example, and in accordance with various embodiments, the ablation catheter 320 preferably has a diameter of about 4 Fr. or less, and the delivery sheath 310 preferably has a diameter of about 4 Fr. or less. The ablation catheter 320 may be configured as an infusion catheter.

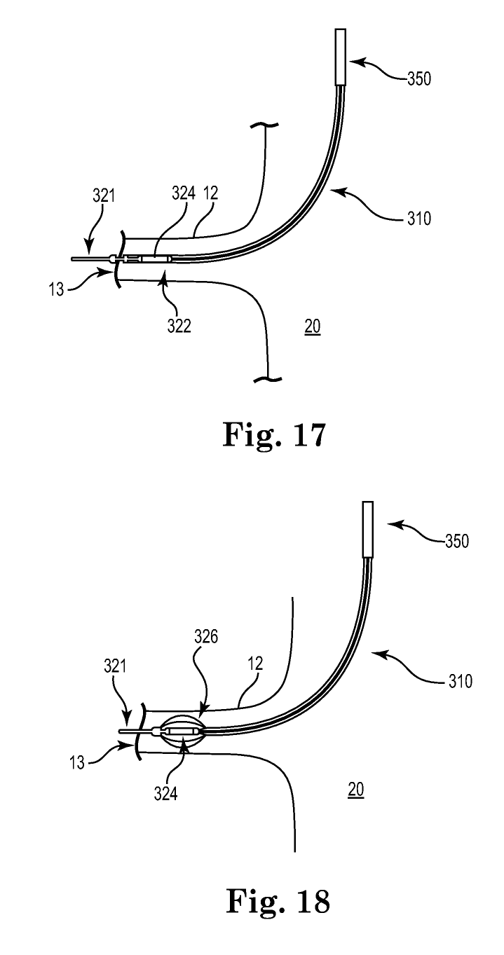

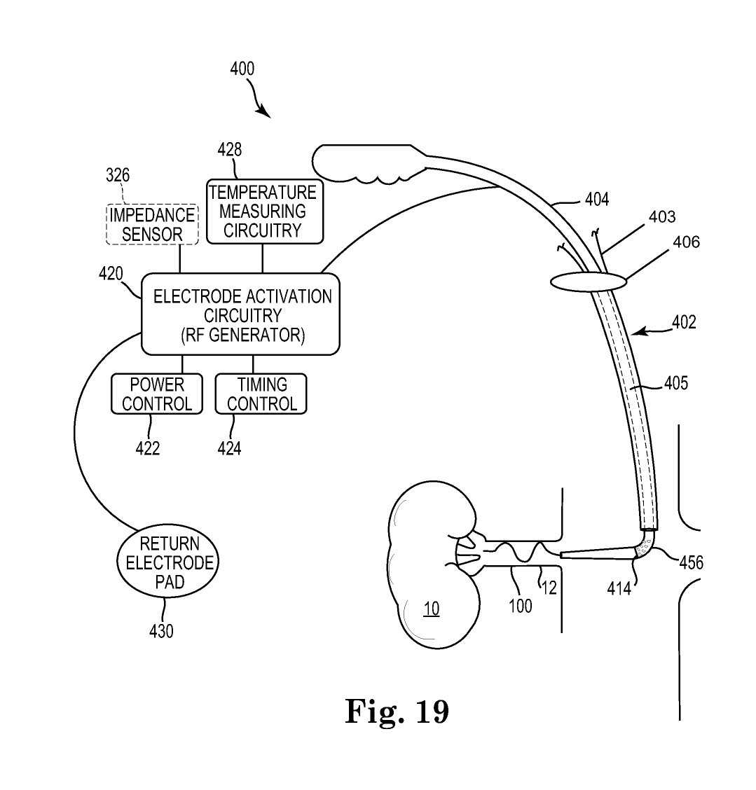

An electrode arrangement 322 is shown provided at a distal end of the ablation catheter 320. The electrode arrangement 322 has a similar construction and functionality as those previously described with regard to FIGS. 12-14, and includes an electrode 324 centered within an expandable centering basket 326 and insulated struts 328. For example, and with reference to FIGS. 17 and 18, the expandable centering basket 326 can be activated in the manner described above by retracting a captured guidewire 321 and basket stop 323 into forced engagement with the centering basket 326. Expanded views of the electrode arrangement 320 in non-deployed and deployed configurations are respectively shown in FIGS. 16A and 16B.

For example, apparatuses in accordance with various embodiments include a small infusion catheter 320 with an ablation region 322 near the distal end of the catheter 320. The ablation region 322 has an RF electrode 224 and a centering basket 326. The ablation region 322 is advanced from either a superior (see FIGS. 17 and 18) or an inferior (see FIG. 15) aortal location into the renal artery 12. The centering basket 322 is expanded to hold the electrode 324 a minimum distance from the renal artery wall to guarantee effective wall cooling from blood flow, and to decrease current density at the artery wall. RF energy provided by an external control unit is passed between the electrode 324 and a ground pad to preferentially ablate perivascular tissue where the target autonomic nerves are located. By positioning the electrode 324 away from the vessel walls, the perivascular tissue is effectively heated while minimizing thermal injury to the renal artery.

A guidewire 321 is provided to aid in positioning the ablation region 322 in the renal artery 12. The guidewire 321 may have limited freedom to move with respect to the ablation catheter 320, so a curved wire tip can be manipulated as needed to gain access to the renal artery 12. The ablation catheter 320 may be configured as an infusion catheter, and can be used for imaging contrast injection.

In the embodiments according to FIGS. 15-18, an external control unit is used to energize the electrode 324 in a controlled manner. Monitoring of the tissue impedance can be used for evaluation of the extent of tissue ablation. RF current passes between the electrode 324 and the ground (such as an external ground pad), passing through the blood for a short distance before passing through the vessel walls and the perivascular tissue. Since blood effectively cools the vessel wall, the target tissue is ablated without injury to the vessel walls. An infusion of fluid through the ablation catheter 320 can locally reduce the conductivity of the blood to reduce current flow directly through the blood so that current preferentially passes through target tissues. A fluid infusion will also reduce effects on the blood and potential fouling of the electrode surface, allowing a smaller electrode to be used.

After ablating renal arterial tissue in the one renal artery, the guidewire 321 is advanced to allow the centering basket 326 to collapse, and the apparatus is repositioned in the contralateral renal artery for treatment. The sheath 310 can be used to further collapse the centering basket 326 if needed. The centering basket 326 can be preferentially closed (“self-collapsing”). Since more effective heating of a larger amount of perivascular tissue is obtained without injury to the renal artery, even bifurcated or multiple renal artery anatomies may be treatable with this approach.

Various embodiments provide for a reduce profile configuration by using a captured guidewire 321. Alternatively, a standard guidewire can be used, by adding an actuation filament or sleeve, with slightly larger profile. For example, a non-conductive filament can pull back on the centering basket stop 323 to deploy the centering basket 326. In other embodiments, a self-expanding basket 326 can be used, and an outer sheath 310 is added. The outer sheath 310 is advanced over the centering basket 326 for low-profile introduction, and is retracted to allow the basket 326 to expand. Other electrode configurations can be used, including multiple electrodes 324 and centering baskets 326 in each renal artery. Spacer configurations other than the illustrated centering basket 326 can be used to keep the electrodes 324 a minimum distance from the artery walls. Instead of a monopolar configuration with a separate ground pad, the ground can be conductive portions of a guide catheter or sheath, or multiple electrodes 324 can be used in a bipolar configuration.

FIG. 19 shows a representative RF renal therapy apparatus 400 in accordance with various embodiments of the disclosure. The apparatus 400 illustrated in FIG. 19 includes external electrode activation circuitry 420 which comprises power control circuitry 422 and timing control circuitry 424. The external electrode activation circuitry 420, which includes an RF generator, is coupled to temperature measuring circuitry 428 and may be coupled to an optional impedance sensor 426. An ablation catheter 402 includes a shaft 404 that incorporates a lumen arrangement 405 configured for receiving a variety of components, such as conductors, pharmacological agents, actuator elements, obturators, sensors, or other components as needed or desired. A delivery sheath 403 may be used to facilitate deployment of the catheter 402 into the arterial system via a percutaneous access site 406 in the embodiment shown in FIG. 19. The distal end of the catheter 402 may include a hinge mechanism 456 to facilitate navigation of the catheter's distal tip around turn of approximately 90° from the aorta to a renal artery 12.

The RF generator of the external electrode activation circuitry 420 may include a pad electrode 430 that is configured to comfortably engage the patient's back or other portion of the body near the kidneys. Radiofrequency energy produced by the RF generator is coupled to the flexible electrode arrangement 100 at the distal end of the ablation catheter 402 by the conductor arrangement disposed in the lumen of the catheter's shaft 404.

Renal denervation therapy using the apparatus shown in FIG. 19 can be performed in a unipolar or monopolar mode using the flexible electrode arrangement 100 positioned within the renal artery 12 and the pad electrode 430 positioned on the patient's back, with the RF generator operating in a monopolar mode. In other implementations, multiple flexible electrode arrangements, such as those shown in previous figures, can be configured for operation in a bipolar configuration, in which case the electrode pad 330 is not needed. Representative bipolar configurations include a pair of flexible electrode arrangements, one in each of the patient's renal arteries. Other representative bipolar configurations include one flexible electrode arrangement positioned in one renal artery and another flexible electrode arrangement positioned in the aorta proximate the aortorenal junction. The radiofrequency energy flows through the flexible electrode arrangement or multiple arrangements in accordance with a predetermined activation sequence (e.g., sequential or concurrent) and into the adjacent tissue of the renal artery. In general, when renal artery or aortal tissue temperatures rise above about 113° F. (50° C.), protein is permanently damaged (including those of renal nerve fibers). If heated over about 65° C. and up to 100° C., cell walls break and oil separates from water. Above about 100° C., tissue desiccates.

According to some embodiments, the electrode activation circuitry 420 is configured to control activation and deactivation of one or more electrodes of the flexible electrode arrangement(s) in accordance with a predetermined energy delivery protocol and in response to signals received from temperature measuring circuitry 428. The electrode activation circuitry 420 controls radiofrequency energy delivered to the electrodes of the flexible electrode arrangement(s) so as to maintain the current densities at a level sufficient to cause heating of the target tissue preferably to a temperature of at least about 55° C.

In some embodiments, one or more temperature sensors are situated at the flexible electrode arrangement(s) and provide for continuous monitoring of renal artery tissue temperatures, and RF generator power is automatically adjusted so that the target temperatures are achieved and maintained. An impedance sensor arrangement 426 may be used to measure and monitor electrical impedance during RF denervation therapy, and the power and timing of the RF generator 420 may be moderated based on the impedance measurements or a combination of impedance and temperature measurements. The size of the ablated area is determined largely by the size, shape, number, and arrangement of the electrodes supported by the flexible electrode arrangement(s), the power applied, and the duration of time the energy is applied.

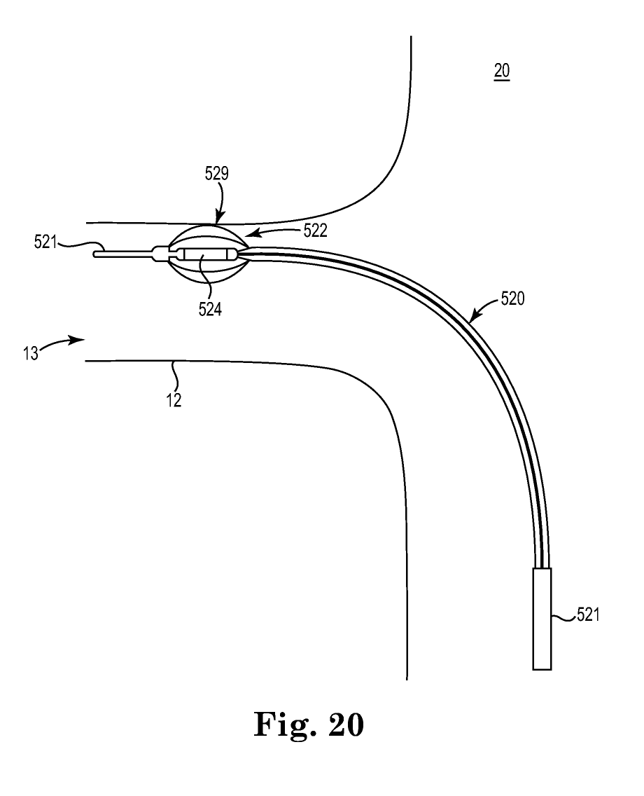

With reference to FIG. 20, there is shown an embodiment of an ablation catheter 520 configured for ablating renal nerves using either a unipolar configuration or a bipolar configuration. In the embodiment shown in FIG. 20, an electrode arrangement 522 is provided at a distal end of the catheter 520 and is encompassed by a spacing basket 529. The spacing basket 529, unlike the centering basket implementations discussed previously, is dimensioned to be smaller than the diameter of the lumen 13 of the renal artery 12. In this configuration, the electrode 524 is preferably positioned at an off-center location within the lumen 13 and biased against an inner wall portion of the renal artery 12.

In use, the ablation catheter 520 is advanced into the renal artery 12 via a delivery sheath 521. When positioned at a desired location within the renal artery 12, the spacing basket 529 is expanded to hold the electrode 524 a desired distance from the renal artery wall, such as between about 0.5 and 1.0 mm away from the renal artery wall. A biasing force produced by the shaft of the catheter 520, which can be augmented by adjusting the position of delivery sheath 521 relative to the catheter's shaft, maintains the expanded spacing basket 529 and electrode 524 in proper position during ablation. The spacing basket 529 can be moved circumferentially about the inner wall of the renal artery 12 to create a circumferential lesion with reduced injury to the renal artery's inner wall. The spacing basket 529, although biased against the wall of the renal artery 12, maintains the electrode 524 at a predefined distance from the artery wall during ablation, which provides effective cooling from blood flow and decreases current density at the artery wall. Biasing, bending, or deflection structures can be provided to bias the spacing basket 529 toward the artery wall as desired. Various aspects of a centered larger-basket device as shown in the figures can be applied to the non-centered smaller basket configurations.

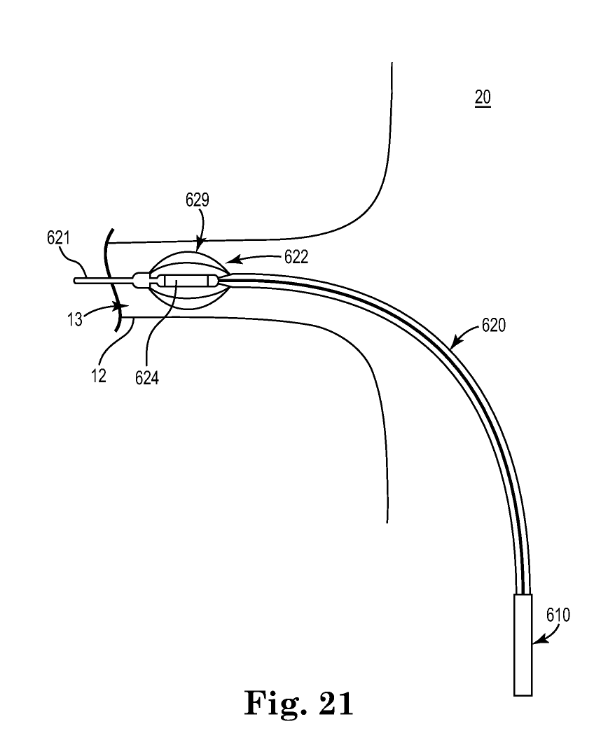

FIG. 21 shows an embodiment of an ablation catheter 620 deployed within the lumen 13 of a patient's renal artery 12. In this embodiment, the ablation catheter 620 includes a centering basket 629 which encompasses an ultrasound ablation device 624. The centering basket 629 is preferably configured in a manner previously described. The ultrasound ablation device 624 preferably includes one or more cylindrical ultrasound transducers which can focus acoustic energy at target tissue and at desired depths within and beyond (e.g., perivascular space) the wall of the renal artery 12. In some embodiments, the ultrasound ablation device 624 can operate at cooler temperatures than RF ablation electrodes due to its ability to focus acoustic energy efficiently at target tissue, which reduces the risk of injury to the inner wall of the renal artery 12.

Representative examples of ultrasound transducers configured for renal denervation are disclosed in commonly owned co-pending U.S. patent application Ser. No. 13/086,116, which is incorporated herein by reference. For example, ultrasound ablation device 624 can be configured as a multiple element intraluminal ultrasound cylindrical phased array, with a multiplicity of ultrasound transducers distributed around the periphery of a cylindrical member. The ultrasound ablation device 624 may be used for imaging and ablation when operated in an imaging mode and an ablation mode, respectively. In some embodiments, renal ablation using the ultrasound ablation device 624 may be conducted under magnetic resonance imaging guidance.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vasculature (e.g., cardiac and urinary vasculature and vessels), and other tissues of the body, including various organs.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter;
a conductor arrangement provided along the catheter;
at least one electrode provided at a distal end of the catheter and in communication with the conductor arrangement; and
a spacing structure provided at the distal end of the catheter and encompassing the at least one electrode, the spacing structure configured to transform between a low-profile introduction configuration and a larger-profile deployed configuration, the spacing structure further configured to maintain space between the at least one electrode and a body vessel, chamber, cavity, or tissue structure when electrical energy sufficient to ablate target tissue adjacent the body vessel, chamber, cavity, or tissue structure is delivered by the at least one electrode.

2. The apparatus of claim 1, wherein the spacing structure comprises perforations allowing for passage of a body fluid therethrough and transport of high frequency AC energy from the at least one electrode to the body vessel, chamber, cavity, or tissue structure via the body fluid.

3. The apparatus of claim 1, wherein the catheter is configured as an infusion catheter through which a fluid can be transported.

4. The apparatus of claim 3, wherein the fluid facilitates one or more of reducing electrical conductivity of surrounding body fluid, reducing fouling of a surface of the at least one electrode by surrounding body fluid, cooling tissue adjacent the at least one electrode, or comprises imaging contrast media.

5. The apparatus of claim 1, wherein the spacing structure comprises a flexible self-collapsing structure.

6. The apparatus of claim 1, wherein the spacing structure comprises a shape-memory member or a superelastic member configured to assume a desired shape.

7. The apparatus of claim 1, wherein the spacing structure comprises a basket structure, and the at least one electrode is situated within the basket structure.

8. The apparatus of claim 1, wherein:
the spacing structure comprises a proximal end and a distal end;
one of the proximal end and distal end of the spacing structure is fixedly mounted to the catheter; and
the other of the proximal end and distal end of the spacing structure is movably mounted to the catheter.

9. The apparatus of claim 10, wherein the catheter comprises an open lumen dimensioned to receive a guidewire, the guidewire comprising a stop member mounted at a distal end of the guidewire, whereby retraction of the guidewire urges the stop member forcibly against the movably mounted distal end of the spacing structure causing axial shortening and radial expansion of the spacing structure.

10. The apparatus of claim 1, further comprising a sheath dimensioned to receive the apparatus, wherein advancement and retraction of the sheath relative to the spacing structure respectively causes collapsing and expansion of the spacing arrangement.

11. The apparatus of claim 1, wherein the spacing structure is configured to center the at least one electrode within a body vessel when in the deployed configuration.

12. The apparatus of claim 1, wherein the spacing structure is configured to position the at least one electrode at an off-center location within a body vessel when in the deployed configuration.

13. The apparatus of claim 1, comprising an external control unit electrically coupled to the at least one electrode and configured to supply energy to the at least one electrode.

14. The apparatus of claim 1, wherein the body vessel, chamber, cavity, or tissue structure comprises a renal artery.

15. An apparatus, comprising:
a first ablation apparatus configured for placement within a first renal artery;
a second ablation apparatus configured for placement within a second renal artery, each of the first and second ablation apparatuses comprising:
a catheter;
a conductor arrangement provided along the catheter;
at least one electrode provided at a distal end of the catheter and in communication with the conductor arrangement; and
a spacing structure provided at the distal end of the catheter and encompassing the at least one electrode, the spacing structure configured to transform between a low-profile introduction configuration and a larger-profile deployed configuration and further configured to maintain space between the at least one electrode and a wall of the respective first and second renal arteries when in the deployed configuration;
wherein each of the at least one electrode of the first and second ablation apparatuses cooperate as a bipolar electrode arrangement for delivering high frequency alternating current sufficient to ablate perivascular renal nerve tissue adjacent the first and second renal arteries and ganglia located at or near first and second aortorenal junctions.

16. The apparatus of claim 15, comprising a sheath having a lumen dimensioned to receive the first and second ablation apparatuses and a length sufficient to deliver the first and second ablation apparatuses to a location at or proximate the first and second renal arteries.

17. The apparatus of claim 15, wherein the catheter of each of the first and second ablation apparatuses is configured as an infusion catheter through which a fluid can be transported.

18. The apparatus of claim 17, wherein the fluid facilitates one or more of reducing electrical conductivity of blood flowing near each of the at least one electrode, reducing fouling of a surface of each of the at least one electrode by surrounding blood, cooling renal artery wall tissue adjacent each of the at least one electrode, or comprises imaging contrast media.

19. The apparatus of claim 15, wherein the spacing structure comprises a flexible self-collapsing structure.

20. The apparatus of claim 15, wherein the spacing structure comprises a shape-memory member or a superelastic member configured to assume a desired shape.

21. The apparatus of claim 15, wherein each spacing structure comprises a basket structure, and the at least one electrode is situated within the basket structure.

22. The apparatus of claim 15, wherein each spacing structure is configured to center the at least one electrode within the respective first and second arteries when in the deployed configuration.

23. The apparatus of claim 15, comprising an external control unit electrically coupled to the at least one electrode of each of the first and second ablation apparatuses and configured to supply energy to each of the at least one electrode in accordance with a predefined activation protocol.

24. A method, comprising:
for each of a patient's renal arteries:
causing a support structure of an ablation apparatus situated within the artery to transform between a low-profile introduction configuration and a larger-profile deployed configuration; and
positioning an electrode of the ablation apparatus within the artery but spaced apart from a wall of the artery using the support structure in the deployed configuration;
ablating perivascular renal nerve tissue adjacent the renal arteries and ganglia located at or near the patient's aortorenal junctions using the electrodes in a bipolar configuration while the support structures are in the deployed configuration; and
causing the support structures to transform from the larger-profile deployed configuration to the low-profile introduction configuration after ablation.

25. The method of claim 24, comprising transporting a fluid through the ablation apparatus, the fluid facilitating one or more of reducing electrical conductivity of blood flowing near the electrodes, reducing fouling of a surface of the electrodes, cooling wall tissue of the renal arteries, or comprising imaging contrast media.

26. The method of claim 24, wherein positioning the electrode comprises positioning the electrode at a center location within the artery when the support structure is in the deployed configuration.

27. The method of claim 24, wherein positioning the electrode comprises positioning the electrode at an off-center location within the artery when the support structure is in the deployed configuration.
